SEQUENCE LISTING (9 sequences)

SEQ ID NO: 1 - DNA, 1271 bp, Homo sapiens
  CDS (193)..(966)
aactttgagatagaagagtacccgctggctgcccagggctttgactgggagctgtggtgccgctacctaaatccccccaaggcctggtggaagctggagaactccacagcgccaatcaggagccctgcccacattggctgcttcattgtggaccggcagtacttccaggagatcggcctgctggacgaaggcatggaagtctacgggggcgagaatgtggagcttgggatcagggtgtggcagtgtggcgggagtgtggaggtcctgccctgctcacggattgcccacattgagcgagcccacaagccctacacagaggacctcaccgcccatgtccgcaggaacgctctcagggtggctgaagtctggatggatgaatttaaaagccacgtctacatggcatggaacataccgcaggaggactcaggaattgacattggggacatcactgcaaggaaggctctcaggaaacagctgcagtgcaagaccttccggtggtacctggtcagcgtgtacccagagatgaggatgtactccgacatcattgcctatggagtgctgcagaattctctgaagactgatttgtgtcttgaccaggggccagatacagagaatgtccccatcatgtacatctgccatgggatgacgcctcagaacgtgtactacacgagcagtcagcagatccatgtgggcattctgagccccaccgtggatgatgatgacaaccgatgcctggtggacgtcaacagccggccccggctcatcgaatgcagctacgccaaagccaagaggatgaagcttcgctggcagttctctcagggaggacccatccagaaccgcaagtctaagcgctgtctggagctgcaggagaatagcgacctggagttcggcttccagctggtgttgcagaagtgctcgggccagcactggagcatcaccaacgtcctgaggagcctcgcgtcctgacccaccggggccacttccggctgcctctttgctactgtgtagcacctgctgcaacgttgcctgctgtccacgtggggttgtttggagtctggggaaccaggttagtgggcccccaagaagagctttttatttcctattcaattttcatggagtttatagaaagatgctgattggtaggtgatggtatgatatcaaactattttgcagttgtaaataggggacagatggaaaatatttataactgacaataaaatattattaagaaaaggaaaaaaaaaaaaacatgtcggccgcctcggcctatg

SEQ ID NO: 2 - protein, 257 aa, Homo sapiens
MEVYGGENVELGIRVWQCGGSVEVLPCSRIAHIERAHKPYTEDLTAHVRRNALRVAEVWMDEFKSHVYMAWNIPQEDSGIDIGDITARKALRKQLQCKTFRWYLVSVYPEMRMYSDIIAYGVLQNSLKTDLCLDQGPDTENVPIMYICHGMTPQNVYYTSSQQIHVGILSPTVDDDDNRCLVDVNSRPRLIECSYAKAKRMKLRWQFSQGGPIQNRKSKRCLELQENSDLEFGFQLVLQKCSGQHWSITNVLRSLAS

SEQ ID NO: 3 - protein, 24 aa, Artificial
  oligonucleotide primer
AACTTTGAGATAGAAGAGTACCCG

SEQ ID NO: 4 - DNA, 24 bp, Artificial
  oligonucleotide primer
cataggccgaggcggccgacatgt

SEQ ID NO: 5 - DNA, 33 bp, Artificial
  oligonucleotide primer
ccccatatgatggaagtctacgggggcgagaat

SEQ ID NO: 6 - DNA, 33 bp, Artificial
  oligonucleotide primer
catggatcctcaggacgcgaggctcctcaggac

SEQ ID NO: 7 - protein, 15 aa, Artificial
  partial sequence of SEQ ID NO 2
MEVYGGENVELGIRV

SEQ ID NO: 8 - DNA, 41 bp, Artificial
  oligonucleotide primer
tggaagtctacgggggcgagaatgtggagcttgggatcagg

SEQ ID NO: 9 - DNA, 41 bp, Artificial
  oligonucleotide primer
tggaagtctacgggggcgagcatgtggagcttgggatcagg

(19) United States
(12) Patent Application Publication
(10) Pub. No.: US 2004/0033505 A1
(43) Pub. Date: Feb. 19, 2004

(54) Novel peptide, an n-acetylgalactosamine transferase-28 and the polynucleotide encoding polypeptide

(76) Inventors: Yumin Mao, Shanghai (CN); Yi Xie, Shanghai (CN)

Correspondence Address: CROWELL & MORING LLP, INTELLECTUAL PROPERTY GROUP, P.O. BOX 14300, WASHINGTON, DC, 20044-4300, US

(21) Appl. No.: 10/276,723

(22) PCT Filed: May 14, 2001
(86) PCT No.: PCT/CN01/00749

(30) Foreign Application Priority Data
May 16, 2000 (CN) 00115726.4

(51) Int. Cl. C12Q 1/68; C07H 21/04; C12N 9/10; C12P 21/02; C12N 5/06; C07K 16/40

(52) U.S. Cl. 435/6; 435/69.1; 435/193; 435/320.1; 435/325; 536/23.2

(57) ABSTRACT

A novel polypeptide, an N-acetylgalactosamine transferase-28, the polynucleotide encoding the polypeptide and the method for producing the polypeptide by DNA recombinant technology. The invention also discloses the uses of the polypeptide in methods for treating various diseases, such as malignant tumour, hemopathy, developmental disorder, HIV infection, immunological disease, and various inflammations, etc. The invention also discloses the agonists against the polypeptide and the therapeutic action thereof. The invention also discloses the uses of the polynucleotide encoding the novel N-acetylgalactosamine transferase-28.

### FIELD OF INVENTION

The invention relates to the field of biotechnology. In particular, the invention relates to a novel polypeptide, N-acetylgalactosamine transferase-28, and a polynucleotide encoding polypeptide. The invention also relates to methods for the preparation and use of said polynucleotide and polypeptide.

### TECHNICAL BACKGROUND

O-glycosidic linkage linked oligosaccharides (mucoprotein-type) are present in various glycoproteins (Sadler, 1984, Biology of Carbohydrates Vol.2, pp.199-213, John Wiley & Sons). The functions of the oligosaccharides vary greatly, ranging from highly specific cell-cell recognition, and host-pathogen inter-reaction, to the more general functions such as preventing proteins from hydrolysis or providing suitable charge and H₂O environment for mucus proteins (Jentoft, 1990, Trends Biochem. Sci. 15:291-294).

The initiation reaction linking oligosaccharide to protein through O-glycosidic linkage is the transfer of an N-acetylgalactosamine group from UDP-N-acetylgalactosamine to a serine or a threonine residue of a protein. The enzyme for this reaction is N-acetylgalactosamine transferase (UDP-GalNAc). It is an intracellular enzyme in the secretory pathway.

Recently biologists have cloned UDP-GalNAc from cattle. This enzyme is a type-II membrane protein. Other common domains of galactosamine transferase also exist in this enzyme. Its amino acid sequence has three consensus sequences as N-linking site. UDP-GalNAc is soluble in water because it lacks a membrane-binding sequence of 40 amino acids in the N-terminus and a fragment which contains a cytoplasmic domain and a transmembrane domain (Homa et al., 1993, J. Biol. Chem., 268:12609-12616).

The precise localization of UDP-GalNAc remains controversial. Some scientists think that the initiative combination of N-acetylgalactose to acceptor protein occurs in pre-rough endoplasmic reticulum, but others think that the combination occurs post-translationally in post-endoplasmic reticulum components or cis-Golgi's. There is evidence that the transferring of N-acetylgalactosamine group to serine or threonine may occur in several isolated areas in the secretory pathway (Schachter, H., and Brockhausen, I., 1992, Marce L. Dekker, Inc., New York 263-332).

The human polypeptide of the present invention shares 34% identity and 58% similarity with UDP-GalNAc at the amino acid sequence level. It has similar structural characteristics to UDP-GalNAc. This human polypeptide, named N-acetylgalactosamintransferase-28, belongs to the UDP-GalNAc family. It is believed to have similar biological functions as UDP-GalNAc.

As above-described, N-acetylgalactosamine transferase-28 plays an essential role in the regulation of important biological functions such as cell division and embryogenesis. It is believed that many proteins are involved. So the determination of related proteins, such as human N-acetylgalactosamine transferase-28, especially of their amino acid sequences, is always desired. The isolation of the novel N-acetylgalactosamine transferase-28 builds the basis for research of the protein function under normal and clinical conditions and this protein can be the basis of disease diagnosis and/or drug development. So the isolation of its cDNA is very improtant.

### OBJECTIVE OF THE INVENTION

One objective of the invention is to provide an isolated novel polypeptide, i.e., N-acetylgalactosamine transferase-28, and fragments, analogues and derivatives thereof.

Another objective of the invention is to provide a polynucleotide encoding said polypeptide.

Another objective of the invention is to provide a recombinant vector containing a polynucleotide encoding an N-acetylgalactosamine transferase-28.

Another objective of the invention is to provide a genetically engineered host cell containing a polynucleotide encoding an N-acetylgalactosamine transferase-28.

Another objective of the invention is to provide a method for producing an N-acetylgalactosamine transferase-28.

Another objective of the invention is to provide an antibody against an N-acetylgalactosamine transferase-28 of the invention.

Another objective of the invention is to provide mimetics, antagonists, agonists, and inhibitors for the polypeptide of the N-acetylgalactosamine transferase-28.

Another objective of the invention is to provide a method for the diagnosis and treatment of diseases associated with an abnormality of N-acetylgalactosamine transferase-28.

### SUMMARY OF INVENTION

The present invention relates to an isolated polypeptide, which is originated from human, and comprises a polypeptide having the amino acid sequence of SEQ ID NO: 2, or its conservative mutants, or its active fragments, or its active derivatives and its analogues. Preferably, the polypeptide has the amino acid sequence of SEQ ID NO: 2.

The present invention also relates to an isolated polynucleotide, comprising a nucleotide sequence or its variant selected from the group consisting of:

(a) a polynucleotide encodeing a polypeptide comprising the amino acid sequence of SEQ ID NO.2;

(b) a polynucleotide complementary to the polynucleotide (a);

(c) a polynucleotide having at least 70% homology to the polynucleotide (a) or (b).

Preferably, said nucleotide sequence is selected from the group consisting of (a) the sequence of position 193-966 in SEQ ID NO: 1; and (b) the sequence of position 1-1271 in SEQ ID NO: 1.

The invention also includes: a vector containing the polynucleotides of said invention, especially an expression vector; a host cell genetically engineered with the vector; which the host cell may be produced by transformation, transduction or transfection; a method for the production of the polypeptide through the process of host cell cultivation and expression product harvest.

The present invention also includes antibodies which could specifically bind the polypeptide.

The invention also includes a method for the selection of compounds which could stimulate, activate, antagonize, or repress the activity of the N-acetylgalactosamine transferase-28, and the compounds obtained by the method.

The invention also includes a method for in vitro assay of the diseases or disease susceptibility related to the abnormal expression of N-acetylgalactosamine transferase-28. The method involves detection of mutation in the polypeptide or its encoding polynucleotide sequence, or the quantitative determination or biological activity assay of the polypeptide in biological samples.

The invention also includes a pharmaceutical composition which comprises the polypeptide, its mimetic, its agonist, its antagonist, its repressor, and a pharmaceutically acceptable carrier.

The invention also includes application of said invented polypeptide and/or its polynucleotide for drug development to treat cancers, developmental diseases, immune diseases, or other diseases caused by abnormal expression of N-acetylgalactosamine transferase-28.

Other aspects of the invention are apparent to the skilled in the art in view of the disclosure.

### BRIEF DESCRIPTION OF THE DRAWINGS

The following drawings are provided to illustrate the embodiment of the invention, not to limit the scope of invention defined by the claims.

FIG. 1 shows an alignment comparison of amino acid sequences of N-acetylgalactosamine transferase-28 of the invention and the N-acetylgalactosamine transferase. The upper sequence is N-acetylgalactosamine transferase-28, and the lower sequence is N-acetylgalactosamine transferase. The identical and similar amino acids are indicated by a one-letter code of amino acid and “+” respectively.

FIG. 2 shows the SDS-PAGE of the isolated N-acetylgalactosamine transferase-28 which has a molecular weight of 28 kDa. The isolated protein band is marked with an arrow.

### DESCRIPTION OF INVENTION

The terms used in this specification and claims have the following meanings, except for the special descriptions.

“Nucleotide sequence” refers to oligonucleotide, nucleotide, or polynucleotide and parts of polynucleotide. It also refers to genomic or synthetic DNA or RNA, which could be single stranded or double stranded, and could represent the sense strand or the antisense strand. Similarly, the term “amino acid sequence” refers to oligopeptide, peptide, polypeptide, or protein sequence and parts of proteins. When the “amino acid sequence” in said invention is related to the sequence of a natural protein, the amino acid sequence of said kind of “peptide” or “protein” will not be limited to be identical to the sequence of that natural protein.

“Variant” of a protein or polynucleotide refers to an amino acid sequence with one or several amino acid changed, or a polynucleotide sequence with one or several nucleotides changed. Such changes include deletion, insertion, or substitution of amino acids in the animo acid sequence, or of nucleotides in the polynucleotide sequence. These changes could be conservative and the substituted amino acid has similar structure or chemical characteristics as the original one, just as the substitution of Ile with Leu. Changes also could be not conservative, just as the substitution of Ala with Trp.

“Deletion” refers to the deletion of one or several amino acids in an amino acid sequence, or of one or several nucleotides in a nucleotide sequence.

“Insertion” or “addition” refers to the addition of one or several amino acids in an amino acid sequence, or of one or several nucleotides in a nucleotide sequence, when compared to the natural molecule. “Substitution” refers to the change of one or several amino acids, or of one or several nucleotides, into different ones without changing the length of the molecule.

“Biological activity” refers to the regulatory or biochemical functions of a protein. Similarly, the term “immunologic activity” refers to the ability of natural, recombinant, or synthetic proteins to induce a specific immunologic reaction, or of binding to a specific antibody in a suitable animal or cell.

“Agonist” refers to the kind of molecule which could up-regulate the activity of N-acetylgalactosamine transferase-28 by binding or changing it. Agonists involve proteins, nucleotides, carbohydrates or any other molecules which could binding the N-acetylgalactosamine transferase-28.

“Antagonist” or “repressor” refers to molecules which could repress or downregulate the biological activity or immune activity of N-acetylgalactosamine transferase-28 when binding it. Antagonists or repressors include proteins, nucleotides, carbohydrates or any other molecules which could bind the N-acetylgalactosamine transferase-28.

“Regulation” refers to changes in the function of N-acetylgalactosamine transferase-28, including increase or decrease of the activity, changes in binding specifity, changes of any other biological characteristics, functional or immune characteristics of N-acetylgalactosamine transferase-28.

“Substantially pure” refers to the condition of purity without any other naturally related proteins, lipids, saccharides, or other substances. An ordinarily skilled person can purify N-acetylgalactosamine transferase-28 by standard protein purification techniques. Substantially pure N-acetylgalactosamine transferase-28 produce a single main band in denaturing polyacrylamide gel electrophoresis. The purity of N-acetylgalactosamine transferase-28 can be analyzed by amino acid sequence analysis.

“Complementary” or “complementation” refers to the natural binding of polynucleotides by base pairing under the condition of suitable ion concentration and temperature. For instance, the sequence “5′-C-T-G-A-3'” could bind to its complementary sequence “3′-G-A-C-T-5′.” The complementation between two single stranded molecules could be partial or complete. Complementary degree between two single strands has obvious influence on the efficiency of hybrid formation and intensity of the hybrid formed.

“Homology” refers to the complementary degree which may be partially or completely homologous. “Partial homology” refers to partially complementary sequence compared to a target nucleotide and the sequence could at least partially repress the hybridization between a completely complementary sequence and the target nucleotide. Repression of the hybridization could be assayed by hybridization (Southern blot or Northern blot) under a lower stringency condition. Substantially complementary sequence or hybrid probe could compete with the completely complementary sequence and repress its hybridization with the target sequence under a lower stringency condition. This does not mean that nonspecific binding is allowed under a lower stringency condition, because specific or selective reaction is still required for hybridization under a lower stringency condition.

“Percent Identity” refers to the percentage of sequence identity or similarity when two or several amino acid or nucleotide sequences are compared. Percent Identity could be determined by computation method such as MEGALIGN program (Lasergene software package, DNASTAR, Inc., Madison Wis.). MEGALIGN program can compare two or several sequences with different methods such as the Cluster method (Higgins, D. G. and Sharp, P. M., 1988, Gene 73:237-244). Cluster method examines the distance between all pairs and arranges the sequences into clusters. Then the clusters are partitioned by pair or group. The identity percentage between two amino acid sequences such as sequence A and B can be calculated by the following equation:

Number of paired residues between sequence A and B/Residue number of sequence A—number of spacing residues in sequence A—number of spacing residue in sequence B×100

Percent Identity between nucleotide sequences can also be determined by Cluster method or other well-known methods in this field such as the Jotun Hein method (Hein J., 1990, Methods in Emzymology, 183:625-645)

“Similarity” refers to the identical degree or conservative substitution degree of amino acid residues in corresponding sites of the amino acid sequences compared to each other. Amino acids for conservative substitution are: negative charged amino acids including Asp and Glu; positive charged amino acids including Leu, Ile and Val; Gly and Ala; Asn and Gln; Ser and Thr; Phe and Tyr.

“Antisense” refers to the nucleotide sequences complementary to a specific DNA or RNA sequence. “Antisense strand” refers to the nucleotide strand complementary to the “sense strand.”

“Derivative” refers to HFP or the chemically modified nucleotide encoding it. This kind of modified chemical can be derived from replacement of the hydrogen atom with an alkyl, acyl, or amino group. The nucleotide derivative can encode peptide retaining the major biological characteristics of the natural molecule.

“Antibody” refers to the intact antibody or its fragments such as Fa, F(ab′)₂ and Fv, and it can specifically bind antigenic determinants of N-acetylgalactosamine transferase-28.

“Humanized antibody” refers to the antibody which has its amino acid sequence in non-antigen binding region replaced to mimic human antibody and still retain the original binding activity.

The term “isolated” refers to the removal of a material out of its original environment (for instance, if it is naturally produced, the original environment refers to its natural environment). For example, a natural produced polynucleotide or a peptide in a living organism means it has not been “isolated.” While the separation of the polynucleotide or a peptide from its coexisting materials in natural system means it was “isolated.” This polynucleotide may be a part of a vector. This polynucleotide or peptide may also be part of a compound. Since the vector or compound is not part of its natural environment, the polynucleotide or peptide is still “isolated.”

As used herein, the term “isolated” refers to a substance which has been isolated from the original environment. For naturally occurring substance, the original environment is the natural environment. For example, the polynucleotide and polypeptide in a naturally occurring state in the viable cells are not isolated or purified. However, if the same polynucleotide and polypeptide have been isolated from other components naturally accompanying them, they are isolated or purified.

As used herein, “isolated N-acetylgalactosamine transferase-28” means that N-acetylgalactosamine transferase-28 does not essentially contain other proteins, lipids, carbohydrate or any other substances associated therewith in nature. Those skilled in the art can purify N-acetylgalactosamine transferase-28 by standard protein purification techniques. The purified polypeptide forms a single main band on a non-reducing PAGE gel. The purity of N-acetylgalactosamine transferase-28 can be analyzed by amino acid sequence analysis.

The invention provides a novel polypeptide—N-acetylgalactosamine transferase-28, which comprises the amino acid sequence shown in SEQ ID NO: 2. The polypeptide of the invention may be a recombinant polypeptide, natural polypeptide, or synthetic polypeptide, preferably a recombinant polypeptide. The polypeptide of the invention may be a purified natural product or a chemically synthetic product. Alternatively, it may be produced from prokaryotic or eukaryotic hosts, such as bacterial, yeast, higher plant, insect, and mammalian cells, using recombinant techniques. Depending on the host used in the protocol of recombinant production, the polypeptide of the invention may be glycosylated or non-glycosylated. The polypeptide of the invention may or may not comprise the starting Met residue.

The invention further comprises fragments, derivatives and analogues of N-acetylgalactosamine transferase-28. As used in the invention, the terms “fragment,” “derivative” and “analogue” mean a polypeptide that essentially retains the same biological functions or activity of N-acetylgalactosamine transferase-28 of the invention. The fragment, derivative or analogue of the polypeptide of the invention may be (i) one in which one or more of the amino acid residues are substituted with a conserved or non-conserved amino acid residue (preferably a conserved amino acid residue) and such substituted amino acid residue may or may not be one encoded by the genetic code; or (ii) one in which one or more of the amino acid residues are substituted with other residues, include a substituent group; or (iii) one in which the mature polypeptide is fused with another compound, such as a compound to increase the half-life of the polypeptide (for example, polyethylene glycol); or (iv) one in which additional amino acids are fused to the mature polypeptide, such as a leader or secretory sequence or a sequence which is employed for purification of the mature polypeptide or a proprotein sequence. Such fragments, derivatives and analogs are deemed to be within the scope of the skilled in the art from the teachings herein.

The invention provides an isolated nucleic acid or polynucleotide which comprises the polynucleotide encoding an amino acid sequence of SEQ ID NO: 2. The polynucleotide sequence of the invention includes the nucleotide sequence of SEQ ID NO: 1. The polynucleotide of the invention was identified in a human embryonic brain cDNA library. Preferably, it comprises a full-length polynucleotide sequence of 1271 bp, whose ORF (193-966) encodes 257 amino acids. Based on amino acid homology comparison, it is found that the encoded polypeptide is 34% homologous to N-acetylgalactosamine transferase. This novel human N-acetylgalactosamine transferase-28 has similar structures and biological functions to those of the N-acetylgalactosamine transferase.

The polynucleotide according to the invention may be in the forms of DNA or RNA. The forms of DNA include cDNA, genomic DNA, and synthetic DNA, etc., in single stranded or double stranded form. DNA may be an encoding strand or non-encoding strand. The coding sequence for mature polypeptide may be identical to the coding sequence shown in SEQ ID NO: 1, or is a degenerate sequence. As used herein, the term “degenerate sequence” means a sequence which encodes a protein or peptide comprising a sequence of SEQ ID NO: 2 and which has a nucleotide sequence different from the sequence of coding region in SEQ ID NO: 1.

The polynucleotide encoding the mature polypeptide of SEQ ID NO: 2 includes those encoding only the mature polypeptide, those encoding mature polypeptide plus various additional coding sequence, the coding sequence for mature polypeptide (and optional additional encoding sequence) plus the non-coding sequence.

The term “polynucleotide encoding the polypeptide” includes polynucleotides encoding said polypeptide and polynucleotides comprising additional coding and/or non-coding sequences.

The invention further relates to variants of the above polynucleotides which encode a polypeptide having the same amino acid sequence of invention, or a fragment, analogue and derivative of said polypeptide. The variant of the polynucleotide may be a naturally occurring allelic variant or a non-naturally occurring variant. Such nucleotide variants include substitution, deletion, and insertion variants. As known in the art, an allelic variant may have a substitution, deletion, and insertion of one or more nucleotides without substantially changing the functions of the encoded polypeptide.

The present invention further relates to polynucleotides, which hybridize to the herein above-described sequences, that is, there is at least 50% and preferably at least 70% identity between the sequences. The present invention particularly relates to polynucleotides, which hybridize to the polynucleotides of the invention under stringent conditions. As herein used, the term “stringent conditions” means the following conditions: (1) hybridization and washing under low ionic strength and high temperature, such as 0.2× SSC, 0.1% SDS, 60° C.; or (2) hybridization after adding denaturants, such as 50% (v/v) formamide, 0.1% bovine serum/0.1% Ficoll, 42° C.; or (3) hybridization only when the homology of two sequences at least 95%, preferably 97%. Further, the polynucleotides which hybridize to the hereinabove described polynucleotides encode a polypeptide which retains the same biological function and activity as the mature polypeptide of SEQ ID NO: 2

The invention also relates to nucleic acid fragments hybridized with the herein above sequence. As used in the present invention, the length of the “nucleic acid fragment” is at least more than 10 bp, preferably at least 20-30 bp, more preferably at least 50-60 bp, and most preferably at least 100 bp. The nucleic acid fragment can be used in amplification techniques of nucleic acid, such as PCR, so as to determine and/or isolate the polynucleotide encoding N-acetylgalactosamine transferase-28.

The polypeptide and polynucleotide of the invention are preferably in the isolated form, preferably purified to be homogenous.

According to the invention, the specific nucleic acid sequence encoding N-acetylgalactosamine transferase-28 can be obtained in various ways. For example, the polynucleotide is isolated by hybridization techniques well-known in the art, which include, but are not limited to 1) the hybridization between a probe and genomic or cDNA library so as to select a homologous polynucleotide sequence, and 2) antibody screening of expression library so as to obtain polynucleotide fragments encoding polypeptides having common structural features.

According to the invention, DNA fragment sequences may further be obtained by the following methods: 1) isolating double-stranded DNA sequence from genomic DNA; and 2) chemical synthesis of DNA sequence so as to obtain the double-stranded DNA.

Among the above methods, the isolation of genomic DNA is least frequently used. A commonly used method is the direct chemical synthesis of DNA sequence. A more frequently used method is the isolation of cDNA sequence. Standard methods for isolating the cDNA of interest is to isolate mRNA from donor cells that highly express said gene followed by reverse transcription of mRNA to form plasmid or phage cDNA library. There are many established techniques for extracting mRNA and kits are commercially available (e.g. Qiagene). Conventional method can be used to construct cDNA library (Sambrook et al., Molecular Cloning: A Laboratory Manual, Cold Spring Harbor Laboratory. New York, 1989). The cDNA libraries are also commercially available. For example, Clontech Ltd. has various cDNA libraries. When PCR is further used, even an extremely small amount of expression products can be cloned.

Numerous well-known methods can be used for screening for the polynucleotide of the invention from cDNA library. These methods include, but are not limited to, (1) DNA-DNA or DNA-RNA hybridization; (2) the appearance or loss of the function of the marker-gene; (3) the determination of the level of N-acetylgalactosamine transferase-28 transcripts; (4) the determination of protein product of gene expression by immunology methods or the biological activity assays. The above methods can be used alone or in combination.

In method (1), the probe used in the hybridization could be homologous to any portion of polynucleotide of invention. The length of probe is typically at least 10 nucleotides, preferably at least 30 nucleotides, more preferably at least 50 nucleotides, and most preferably at least 100 nucleotides. Furthermore, the length of the probe is usually less than 2000 nucleotides, preferably less than 1000 nucleotides. The probe usually is the DNA sequence chemically synthesized on the basis of the sequence information. Of course, the gene of the invention itself or its fragment can be used as a probe. The labels for DNA probe include, e.g., radioactive isotopes, fluoresceins or enzymes such as alkaline phosphatase.

In method (4), the detection of the protein products expressed by N-acetylgalactosamine transferase-28 gene can be carried out by immunology methods, such as Western blotting, radioimmunoassay, and ELISA.

The method of amplification of DNA/RNA by PCR (Saiki et al. Science 1985; 230:1350-1354) is preferably used to obtain the polynucleotide of the invention. Especially when it is difficult to obtain the full-length cDNA, the method of RACE (RACE- cDNA terminate rapid amplification) is preferably used. The primers used in PCR can be selected according to the polynucleotide sequence information of the invention disclosed herein, and can be synthesized by conventional methods. The amplified DNA/RNA fragments can be isolated and purified by conventional methods such as gel electrophoresis.

Sequencing of polynucleotide sequence of the gene of the invention or its various DNA fragments can be carried out by the conventional dideoxy sequencing method (Sanger et al. PNAS, 1977, 74: 5463-5467). Sequencing of polynucleotide sequence can also be carried out using commercially available sequencing kits. In order to obtain the full-length cDNA sequence, it is necessary to repeat the sequencing process. Sometimes, it is needed to sequence the cDNA of several clones to obtain the full-length cDNA sequence.

The invention further relates to a vector comprising the polynucleotide of the invention, a genetically engineered host cell transformed with the vector of the invention or directly with the sequence encoding N-acetylgalactosamine transferase-28, and a method for producing the polypeptide of the invention by recombinant techniques.

In the present invention, the polynucleotide sequences encoding N-acetylgalactosamine transferase-28 may be inserted into a vector to form a recombinant vector containing the polynucleotide of the invention. The term “vector” refers to a bacterial plasmid, bacteriophage, yeast plasmid, plant virus or mammalian virus such as adenovirus, retrovirus or any other vehicle known in the art. Vectors suitable for use in the present invention include, but are not limited to the T7-based expression vector for expression in bacteria (Rosenberg et al., Gene, 56:125, 1987), The pMSXND expression vector for expression in mammalian cells (Lee and Nathans, J. Biol. Chem., 263:3521, 1988) and baculovirus-derived vectors for expression in insect cells. Any plasmid or vector can be used to construct the recombinant expression vector as long as it can replicate and is stable in the host. One important feature of an expression vector is that the expression vector typically contains an origin of replication, a promoter, a marker gene as well as translation regulatory components.

Methods known in the art can be used to construct an expression vector containing the DNA sequence of N-acetylgalactosamine transferase-28 and appropriate transcription/translation regulatory components. These methods include in vitro recombinant DNA technique, DNA synthesis technique, in vivo recombinant technique and so on (Sambroook et al. Molecular Cloning, a Laboratory Manual, Cold Spring Harbor Laboratory. New York, 1989). The DNA sequence is operatively linked to a proper promoter in an expression vector to direct the synthesis of mRNA. Exemplary promoters are lac or trp promoter of E. coli; PL promoter of A phage; eukaryotic promoters including CMV immediate early promoter, HSV thymidine kinase promoter, early and late SV40 promoter, LTRs of retrovirus, and other known promoters which control gene expression in the prokaryotic cells, eukaryotic cells or viruses. The expression vector may further comprise a ribosome binding site for initiating translation, transcription terminator and the like. Transcription in higher eukaryotes is increased by inserting an enhancer sequence into the vector. Enhancers are cis-acting elements of DNA, usually about from 10 to 300 bp in length that act on a promoter to increase gene transcription level. Examples include the SV40 enhancer on the late side of the replication origin 100 to 270 bp, the polyoma enhancer on the late side of the replication origin, and adenovirus enhancers.

Further, the expression vector preferably comprises one or more selective marker genes to provide a phenotype for the selection of the transformed host cells, e.g., the dehydrofolate reductase, neomycin resistance gene and GFP (green flurencent protein) for eukaryotic cells, as well as tetracycline or ampicillin resistance gene for E. coli.

The skilled in the art know clearly how to select appropriate vectors, transcriptional regulatory elements, e.g., promoters, enhancers, and selective marker genes.

According to the invention, polynucleotide encoding N-acetylgalactosamine transferase-28 or recombinant vector containing said polynucleotide can be transformed or transfected into host cells to construct genetically engineered host cells containing said polynucleotide or said recombinant vector. The term “host cell” means prokaryote, such as bacteria; or lower eukaryote, such as yeast; or higher eukaryotic, such as mammalian cells. Representative examples are bacterial cells, such as E. coli, Streptomyces, Salmonella typhimurium; fungal cells, such as yeast; plant cells; insect cells such as Drosophila S2 or Sf9; animal cells such as CHO, COS or Bowes melanoma.

Transformation of a host cell with the DNA sequence of the invention or a recombinant vector containing said DNA sequence may be carried out by conventional techniques well known to those skilled in the art. When the host is prokaryotic, such as E. coli, competent cells, which are capable of DNA uptake, can be prepared from cells harvested after exponential growth phase and subsequently treated by the CaCl₂ method using procedures well known in the art. Alternatively, MgCl₂ can be used. Transformation can also be carried out by electroporation, if desired. When the host is an eukaryote, transfection methods as well as calcium phosphate precipitation may be used. Conventional mechanical procedures such as micro-injection, electroporation, or liposome-mediated transfection may also be used.

The recombinant N-acetylgalactosamine transferase-28 can be expressed or produced by conventional recombinant DNA technology (Science, 1984; 224:1431), using the polynucleotide sequence of the invention. The steps generally include:

(1) transfecting or transforming appropriate host cells with the polynucleotide (or variant) encoding human N-acetylgalactosamine transferase-28 of the invention or the recombinant expression vector containing said polynucleotide;

(2) culturing the host cells in an appropriate medium; and

(3) isolating or purifying the protein from the medium or cells.

In Step (2) above, depending on the host cells used, the medium for cultivation can be selected from various conventional media. The host cells are cultured under a condition suitable for its growth until the host cells grow to an appropriate cell density. Then, the selected promoter is induced by appropriate means (e.g., temperature shift or chemical induction) and cells are cultured for an additional period.

In Step (3), the recombinant polypeptide may be included in the cells, or expressed on the cell membrane, or secreted out of the cell. If desired, physical, chemical and other properties can be utilized in various isolation methods to isolate and purify the recombinant protein. These methods are well-known to those skilled in the art and include, but are not limited to conventional renaturation treatment, treatment by a protein precipitant (such as salt precipitation), centrifugation, cell lysis by osmosis, sonication, supercentrifugation, molecular sieve chromatography or gel chromatography, adsorption chromatography, ion exchange chromatagraphy, HPLC, and any other liquid chromatagraphy, and a combination thereof.

The polypeptide of the invention and antagonists, agonists and inhibitors thereof can be directly used for the treatment of diseases, e.g., various malignant tumors or cancers, dermatitis, inflammation, adrenoprival disease and HIV infection and immune system diseases.

O-glycosidic linkages linked oligosaccharides (mucoprotein-type) are present in various glycoproteins. The oligosaccharides have various functions. There are highly specific oligosaccharides that take part in cell-cell identification and host-pathogen reaction and common oligosaccharides that prevent proteins from hydrolysis or provide suitable charges or H₂O environment in mucus excretion proteins.

The initiation reaction of the synthesis of O-glycosidic linkage to oligosaccharide needs a catalytic enzyme, UDP-GalNAc, which is an intracellular enzyme in secretory pathway.

The polypeptide of the invention and UDP-GalNAc are members of the UDP-GalNAc family. This polypeptide of the invention has characteristic sequences of the UDP-GalNAc family. They have similar biological functions. The polypeptide participates in the initiation reaction of the synthesis of O-glycosidic linkage links to oligosaccharide in vivo. It is prerequisite for this synthesis. The oligosaccharide linked by O-glycosidic linkage plays an important role in the proper functions of many glycoproteins. Abnormal expression of this polypeptide is usually closely related to abnormal functions of many glycoproteins, thus affects immune regulation, inflammation reaction, cell division etc., and causes various diseases.

Accordingly, the abnormal expression of UDP-GalNAc-28 in this invention will result in many diseases, especially various tumors, embryogenesis disorder, growth and development disorder, inflammation, and immune disease. These include but are not limited to:

Various tissue tumors: carcinoma of stomach, liver cancer, lung cancer, esophagus cancer, mammary cancer, Leukemia, lymphadenoma, struma of thyroid, hysteromyoma, neurocytoma, astrocytoma, ependymocytoma, gliocytoma, neurofibroma, colon cancer, melanotic carcinoma, bladder cancer, cancer of the womb, endometria cancer, thymic carcinoma, carcinoma of nasopharynx, laryngocarcinoma, tracheocarcinoma, fibroid tumor, fibrosarcoma, lipoma, liposarcoma.

Embryogenesis disorder: congenital abortion, cleftpalate, limb defect, limb differential disturbance, interatriale septum defect, neural tube defect, congenital hydrocephelus, congenital glaucoma or cataract, congenital deafness.

Growth and development disorder: mental retardation, encephalo disturbance,dermo fat and muscle dysplasia, bone and arthron dysplasia, various decompensation diseases, cretinism, nanism, Cushing's syndroma, sexual development retardation.

Inflammations: chronic activity hepatitis, nodus disease, polymyositis, chronic rhinitis, chronic gastritis, Cord multiple sclerosis, glomerular nephritis, myocarditis, cardiomyopathy, atheroma, gastric ulce, cervicitis, various infective inflammations.

Immune diseases: systemic lupus erythematosus, atrophic arthritis, bronchial asthma, specific dermatitis, infective myocarditis, scleroderma, myasthenia gravis, Greenk-Baly syndroma, normal inconstancy immunologic deficiency, primary lymphocyte B immunologic deficiency, acquired immunologic deficiency syndroma.

The abnormal expression of UDP-GalNAc-28 in this invention will also result in some heritage diseases, haemal diseases etc.

The polypeptide and its antagonist, agonism, and inhibitor can be used directly to cure diseases, especially various tumors, embryogenesis disorder, growth and development disorder, inflammation, immunity disease, some genetic diseases, and blood diseases etc.

The invention also provides methods for screening compounds so as to identify an agent which enhances N-acetylgalactosamine transferase-28 activity (agonists) or decrease N-acetylgalactosamine transferase-28 activity (antagonists). The agonists enhance the biological functions of N-acetylgalactosamine transferase-28 such as inactivation of cell proliferation, while the antagonists prevent and cure the disorders associated with the excess cell proliferation, such as various cancers. For example, in the presence of an agent, the mammal cells or the membrane preparation expressing N-acetylgalactosamine transferase-28 can be incubated with the labeled N-acetylgalactosamine transferase-28 to determine the ability of the agent to enhance or repress the interaction.

Antagonists of N-acetylgalactosamine transferase-28 include antibodies, compounds, receptor deletants and analogues. The antagonists of N-acetylgalactosamine transferase-28 can bind to N-acetylgalactosamine transferase-28 and eliminate or reduce its function, or inhibit the production of N-acetylgalactosamine transferase-28, or bind to the active site of said polypeptide so that the polypeptide can not function biologically.

When screening for compounds as an antagonist, N-acetylgalactosamine transferase-28 may be added into a biological assay. It can be determined whether the compound is an antagonist or not by determining its effect on the interaction between N-acetylgalactosamine transferase-28 and its receptor. Using the same method as that for screening compounds, receptor deletants and analogues acting as antagonists can be selected. Polypeptide molecules capable of binding to N-acetylgalactosamine transferase-28 can be obtained by screening a polypeptide library comprising various combinations of amino acids bound onto a solid matrix. Usually, N-acetylgalactosamine transferase-28 is labeled in the screening.

The invention further provides a method for producing antibodies using the polypeptide, and its fragment, derivative, analogue or cells as an antigen. These antibodies may be polyclonal or monoclonal antibodies. The invention also provides antibodies against epitopes of N-acetylgalactosamine transferase-28. These antibodies include, but are not limited to, polyclonal antibody, monoclonal antibody, chimeric antibody, single-chain antibody, Fab fragment and the fragments produced by a Fab expression library.

Polyclonal antibodies can be prepared by immunizing animals, such as rabbit, mouse, and rat, with N-acetylgalactosamine transferase-28. Various adjuvants, including but are not limited to Freund's adjuvant, can be used to enhance the immunization. The techniques for producing N-acetylgalactosamine transferase-28 monoclonal antibodies include, but are not limited to, the hybridoma technique (Kohler and Milstein, 1975, Nature, 256:495-497), the trioma technique, the human B-cell hybridoma technique, the EBV-hybridoma technique and so on. A chimeric antibody comprising a constant region of human origin and a variable region of non-human origin can be produced using methods well-known in the art (Morrison et al, 1985, PNAS, 81:6851). Furthermore, techniques for producing a single-chain antibody (U.S. Pat. No. 4,946,778) are also useful for preparing single-chain antibodies against N-acetylgalactosamine transferase-28.

The antibody against N-acetylgalactosamine transferase-28 can be used in immunohistochemical method to detect the presence of N-acetylgalactosamine transferase-28 in a biopsy specimen.

The monoclonal antibody specific to N-acetylgalactosamine transferase-28 can be labeled by radioactive isotopes, and injected into human body to trace the location and distribution of N-acetylgalactosamine transferase-28. This radioactively labeled antibody can be used in the non-wounding diagnostic method for the determination of tumor location and metastasis.

Antibodies can also be designed as an immunotoxin targeting a particular site in the body. For example, a monoclonal antibody having high affinity to N-acetylgalactosamine transferase-28 can be covalently bound to bacterial or plant toxins, such as diphtheria toxin, ricin, ormosine. One common method is to challenge the amino group on the antibody with sulfydryl cross-linking agents, such as SPDP, and bind the toxin onto the antibody by interchanging the disulfide bonds. This hybrid antibody can be used to kill N-acetylgalactosamine transferase-28-positive cells.

The antibody of the invention is useful for the therapy or the prophylaxis of disorders related to the N-acetylgalactosamine transferase-28. The appropriate amount of antibody can be administrated to stimulate or block the production or activity of N-acetylgalactosamine transferase-28.

The invention further provides diagnostic assays for quantitative and in situ measurement of N-acetylgalactosamine transferase-28 level. These assays are well known in the art and include FISH assay and radioimmunoassay. The level of N-acetylgalactosamine transferase-28 detected in the assay can be used to illustrate the importance of N-acetylgalactosamine transferase-28 in diseases and to determine the diseases associated with N-acetylgalactosamine transferase-28 .

The polypeptide of the invention is useful in the analysis of polypeptide profile. For example, the polypeptide can be specifically digested by physical, chemical, or enzymatic means, and then analyzed by one, two or three dimensional gel electrophoresis, preferably by spectrometry.

The novel N-acetylgalactosamine transferase-28 polynucleotides of the invention also have many therapeutic applications. Gene therapy technology can be used in the therapy of abnormal cell proliferation, development or metabolism, which are caused by the loss of N-acetylgalactosamine transferase-28 expression or the abnormal or non-active expression of N-acetylgalactosamine transferase-28. Recombinant gene therapy vectors, such as virus vectors, can be designed to express mutated N-acetylgalactosamine transferase-28 so as to inhibit the activity of endogenous N-acetylgalactosamine transferase-28. For example, one form of mutated N-acetylgalactosamine transferase-28 is a truncated N-acetylgalactosamine transferase-28 whose signal transduction domain is deleted. Therefore, this mutated N-acetylgalactosamine transferase-28 can bind the downstream substrate without the activity of signal transduction. Thus, the recombinant gene therapy vectors can be used to cure diseases caused by abnormal expression or activity of N-acetylgalactosamine transferase-28. The expression vectors derived from a virus, such as retrovirus, adenovirus, adeno-associated virus, herpes simplex virus, parvovirus, and so on, can be used to introduce the N-acetylgalactosamine transferase-28 gene into the cells. The methods for constructing a recombinant virus vector harboring N-acetylgalactosamine transferase-28 gene are described in the literature (Sambrook et al. supra). In addition, the recombinant N-acetylgalactosamine transferase-28 gene can be packed into liposome and then transferred into the cells.

The methods for introducing the polynucleotides into tissues or cells include directly injecting the polynucleotides into tissue in the body; or introducing the polynucleotides into cells in vitro with vectors, such as virus, phage, or plasmid, etc, and then transplanting the cells into the body.

Also included in the invention are ribozyme and the oligonucleotides, including antisense RNA and DNA, which inhibit the translation of the N-acetylgalactosamine transferase-28 mRNA. Ribozyme is an enzyme-like RNA molecule capable of specifically cutting certain RNA. The mechanism is nucleic acid endo-cleavage following specific hybridization of ribozyme molecule and the complementary target RNA. Antisense RNA and DNA as well as ribozyme can be prepared by using any conventional techniques for RNA and DNA synthesis, e.g., the widely used solid phase phosphite chemical method for oligonucleotide synthesis. Antisense RNA molecule can be obtained by the in vivo or in vitro transcription of the DNA sequence encoding said RNA, wherein said DNA sequence is integrated into the vector and downstream of the RNA polymerase promoter. In order to increase its stability, a nucleic acid molecule can be modified in many manners, e.g., increasing the length of two the flanking sequences, replacing the phosphodiester bond with the phosphothioester bond in the oligonucleotide.

The polynucleotide encoding N-acetylgalactosamine transferase-28 can be used in the diagnosis of N-acetylgalactosamine transferase-28 related diseases. The polynucleotide encoding N-acetylgalactosamine transferase-28 can be used to detect whether N-acetylgalactosamine transferase-28 is expressed or not, and whether the expression of N-acetylgalactosamine transferase-28 is normal or abnormal in the case of diseases. For example, N-acetylgalactosamine transferase-28 DNA sequences can be used in the hybridization with biopsy samples to determine the expression of N-acetylgalactosamine transferase-28. The hybridization methods include Southern blotting, Northern blotting and in situ blotting, etc., which are well-known and established techniques. The corresponding kits are commercially available. A part of or all of the polynucleotides of the invention can be used as probe and fixed on a microarray or DNA chip for analysis of differential expression of genes in tissues and for the diagnosis of genes. The N-acetylgalactosamine transferase-28 specific primers can be used in RNA-polymerase chain reaction and in vitro amplification to detect transcripts of N-acetylgalactosamine transferase-28.

Further, detection of mutations in N-acetylgalactosamine transferase-28 gene is useful for the diagnosis of N-acetylgalactosamine transferase-28-related diseases. Mutations of N-acetylgalactosamine transferase-28 include site mutation, translocation, deletion, rearrangement and any other mutations compared with the wild-type N-acetylgalactosamine transferase-28 DNA sequence. The conventional methods, such as Southern blotting, DNA sequencing, PCR and in situ blotting, can be used to detect a mutation. Moreover, mutations sometimes affects the expression of protein. Therefore, Northern blotting and Western blotting can be used to indirectly determine whether the gene is mutated or not.

Sequences of the present invention are also valuable for chromosome identification. The sequence is specifically targeted to and can hybridize with a particular location on an individual human chromosome. There is a current need for identifying particular sites of gene on the chromosome. Few chromosome marking reagents based on actual sequence data (repeat polymorphism) are presently available for marking chromosomal location. The mapping of DNA to chromosomes according to the present invention is an important first step in correlating those sequences with genes associated with disease.

Briefly, sequences can be mapped to chromosomes by preparing PCR primers (preferably 15-35 bp) from the cDNA. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the oligonucleotide primers of the invention, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clones to a metaphase chromosomal spread can be used to provide a precise chromosomal location in one step. For a review of this technique, see Verma et al., Human Chromosomes: a Manual of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis.

Next, it is necessary to determine the differences in the cDNA or genomic sequence between affected and unaffected individuals. If a mutation is observed in some or all of the affected individuals but not in any normal individuals, then the mutation is likely to be the cause of the disease. Comparison of affected and unaffected individuals generally involves first looking for structural alterations in the chromosomes, such as deletions or translocations, that are visible from chromosome level, or detectable using PCR based on that DNA sequence. With current resolution of physical mapping and genetic mapping techniques, a cDNA precisely localized to a chromosomal region associated with the disease could be one of 50 to 500 potential causative genes. (This assumes 1 megabase mapping resolution and one gene per 20 kb).

According to the invention, the polypeptides, polynucleotides and its mimetics, agonists, antagonists and inhibitors may be employed in combination with a suitable pharmaceutical carrier. Such a carrier includes but is not limited to water, glucose, ethanol, salt, buffer, glycerol, and combinations thereof Such compositions comprise a safe and effective amount of the polypeptide or antagonist, as well as a pharmaceutically acceptable carrier or excipient with no influence on the effect of the drug. These compositions can be used as drugs in disease treatment.

The invention also provides a pharmaceutical pack or kit comprising one or more containers filled with one or more of the ingredients of the pharmaceutical compositions of the invention. With such container(s) there may be a notice from a governmental agency, that regulates the manufacture, use or sale of pharmaceuticals or biological products, the notice reflects government's approval for the manufacture, use or sale for human administration. In addition, the polypeptides of the invention may be employed in conjunction with other therapeutic compounds.

The pharmaceutical compositions may be administered in a convenient manner, such as through topical, intravenous, intraperitoneal, intramuscular, subcutaneous, intranasal or intradermal routes. N-acetylgalactosamine transferase-28 is administered in an amount, which is effective for treating and/or prophylaxis of the specific indication. The amount of N-acetylgalactosamine transferase-28 administrated on patient will depend upon various factors, such as delivery methods, the subject health, the judgment of the skilled clinician.

### BEST MODE FOR CARRYING OUT THE INVENTION

The invention is further illustrated by the following examples. It is appreciated that these examples are only intended to illustrate the invention, not to limit the scope of the invention. For the experimental methods in the following examples, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified.

### EXAMPLE 1

### Cloning of the N-acetylgalactosamine Transferase-28 Gene

Total RNA from a human embryonic brain was extracted by the one-step method with guanidinium isocyanate/phenol/chloroform. The poly(A) mRNA was isolated from the total RNA with Quik mRNA Isolation Kit (Qiegene). cDNA was prepared by reverse transcription with 2 μg poly(A) mRNA. The cDNA fragments were inserted into the polyclonal site of pBSK(+) vector (Clontech) using Smart cDNA cloning kit (Clontech) and then transformed into DH5α to form the cDNA library. The 5′- and 3′-ends of all clones were sequenced with Dye terminate cycle reaction sequencing kit (Perkin-Elmer) and ABI 377 Automatic Sequencer (Perkin-Elmer). The sequenced cDNA were compared with the public database of DNA sequences (Genebank) and the DNA sequence of one clone 2105d02 was found to be a novel DNA sequence. The inserted cDNA sequence of clone 2105d02 was dual-directionally sequenced with a series of synthesized primers. It was indicated that the full length cDNA contained in clone 2105d02 was 1271bp (SEQ ID NO: 1) with a 774bp ORF located in positions 193-966 which encoded a novel protein (SEQ ID NO: 2). This clone was named pBS-2105d02 and the encoded protein was named N-acetylgalactosamine transferase-28.

### EXAMPLE 2

### Homology Search of cDNA Clone

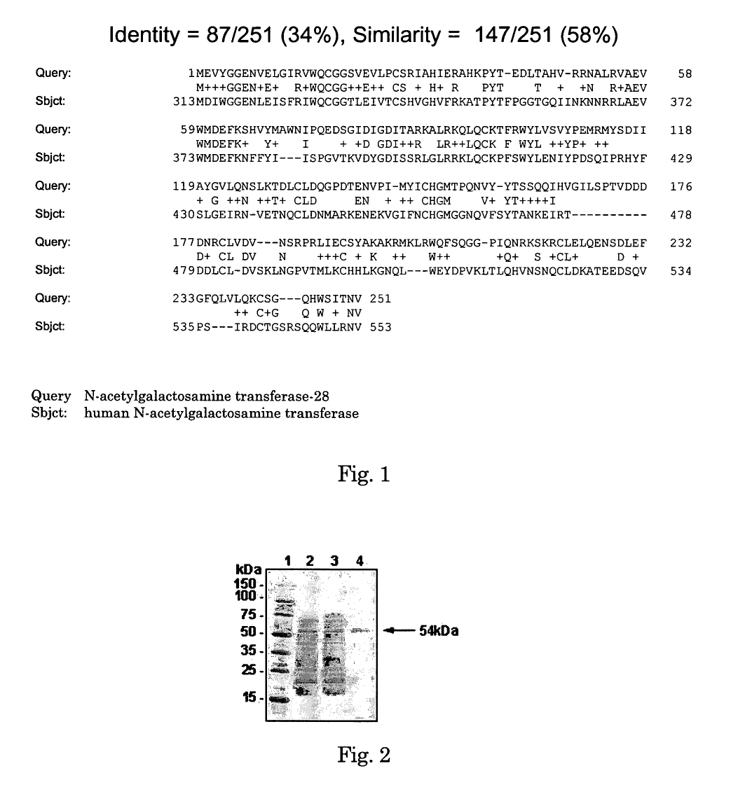

The homology research of the DNA sequence and its protein sequence of N-acetylgalactosamine transferase-28 of the invention were performed by Blast (Basic Local Alignment Search Tool) (Altschul et al., 1990, J. Mol. Biol., 215:403-10) in databases such as Genbank and Swissport. The most homologous gene to N-acetylgalactosamine transferase-28 of the invention is the known N-acetylgalactosamine transferase. The Genbank accession number of its encoded protein is U73820. The alignment result of the protein was shown in FIG. 1. The two proteins are highly homologous with an identity of 34% and a similarity of 58%.

### EXAMPLE 3

### Cloning of N-acetylgalactosamine Transferase-28 Gene by RT-PCR

The template was total RNA extracted from a human embryonic brain. The reverse transcription was carried out with oligo-dT primer to produce cDNAs. After cDNA was purified with Qiagen Kit, PCR was carried out with the following primers:

| | |
|---|---|
| Primer 1: | |
| 5′-AACTTTGAGATAGAAGAGTACCCG-3′ | (SEQ ID NO: 3) |
| Primer 2: | |
| 5′-CATAGGCCGAGGCGGCCGACATGT-3′ | (SEQ ID NO: 4) |

Primer 1 is the forward sequence started from position 1 of 5′ end of SEQ ID NO: 1.

Primer 2 is the reverse sequence of the 3′ end of SEQ ID NO: 1.

The amplification condition was a 50 μl reaction system containing 50 mmol/L KCl, 10 mmol/L Tris-Cl (pH8.5), 1.5 mmol/L MgCl₂,200 umol/L dNTP, 10 pmol of each primer, 1U Taq DNA polymerase(Clontech). The reaction was performed on a PE 9600 DNA amplifier with the following parameters: 94° C. 30 sec, 55° C. 30sec, and 72° C. 2min for 25 cycles. β-actin was used as a positive control, and a blank template, as a negative control in RT-PCR. The amplified products were purified with a QIAGEN kit, and linked with a pCR vector (Invitrogen) using a TA Cloning Kit. DNA sequencing results show that the DNA sequence of PCR products was identical to nucleotides 1-1271bp of SEQ ID NO: 1.

### EXAMPLE 4

### Northern Blotting of Expression of N-acetylgalactosamine Transferase-28 Gene

Total RNA was extracted by one-step method (Anal. Biochem., 1987, 162: 156-159) with guanidinium isocyanate-phenol-chloroform. That is, homogenate the organize using 4M guanidinium isocyanate-25mM sodium citrate, 0.2 sodium acetate (pH4.0), add 1 volume phenol and ⅕ volume chloroform-isoamyl alcohol (49:1), centrifuge after mixing. Take out the aqueous phase, add 0.8 volume isopropyl alcohol, then centrifuge the mixture. Wash the RNA precipitation using 70% ethanol, then dry, then dissolve it in the water. 20μg RNA was electrophoresed on the 1.2% agarose gel containing 20 mM 3-(N-morpholino) propane sulfonic acid (pH 7.0)-5mM sodium acetate-imM EDTA- 2.2M formaldehyde. Then transfer it to a nitrocellulose filter. Prepare the ³²P-labelled DNA probe with α-³²P dATP by random primer method. The used DNA probe is the coding sequence (193bp-966bp) of N-acetylgalactosamine transferase-28 amplified by PCR indicated in FIG. 1. The nitrocellulose filter with the transferred RNA was hybridized with the ³²P-labelled DNA probe (2×10⁶ cpm/ml) overnight in a buffer containing 50% formamide-25 mM KH₂PO₄(Ph7.4)-5×Denhardt's solution and 200 μg /ml salmine. Then wash the filter in the 1× SSC-0.1% SDS, at 55° C., for 30 min. Then analyze and quantitative determinate using Phosphor Imager.

### EXAMPLE 5

### In Vitro Expression, Isolation and Purification of Recombinant N-acetylgalactosamine transferase-28

A pair of primers for specific amplification was designed based on SEQ ID NO: 1 and the encoding region in FIG. 1, the sequences are as follows:

(SEQ ID NO: 5)

| | |
|---|---|
| Primer3: | 5′-CCCCATATGATGGAAGTCTACGGGGGCGAGAAT-3′ |

(SEQ ID NO: 6)

| | |
|---|---|
| Primer4: | 5′-CATGGATCCTCAGGACGCGAGGCTCCTCAGGAC-3′ |

These two primers contain a NdeI and BamHI cleavage site on the 5′ end respectively. Within the sites are the coding sequences of the 5′ and 3′ end of the desired gene. NdeI and BamHI cleavage sites were corresponding to the selective cleavage sites on the expression vector pET-28b(+) (Novagen, Cat. No. 69865.3). PCR amplification was performed with the plasmid pBS-2105d02 containing the full-length target gene as a template. The PCR reaction was subject to a 50 μl system containing 10 pg pBS-2105d02 plasmid, 10 pmol of Primer-3 and 10 pmol of Primer-4, 1 μl of Advantage polymerase Mix (Clontech). The parameters of PCR were 94° C. 20 sec, 60° C. 30 sec, and 68° C. 2 min for 25 cycles. After digesting the amplification products and the plasmid pET-28(+) by NdeI and EcoRI, the large fragments were recovered and ligated with T4 ligase. The ligated product was transformed into E.coli DH5α with the calcium chloride method. After cultured overnight on a LB plate containing a final concentration of 30 μg/ml kanamycin, positive clones were selected out using colony PCR and then sequenced. The positive clone (pET-2105d02) with the correct sequence was selected out and the recombinant plasmid thereof was transformed into BL21(DE3)plySs (Novagen) using the calcium chloride method. In a LB liquid medium containing a final concentration of 30 μg/ml of kanamycin, the host bacteria BL21(pET-2105d02) were cultured at 37° C. to the exponential growth phase, then IPTG were added with the final concentration of 1 mmol/L, the cells were cultured for another 5 hours, and then centrifuged to harvest the bacteria. After the bacteria were sonicated, the supernatant was collected by centrifugation. Then the purified desired protein—N-acetylgalactosamine transferase-28 was obtained by a His.Bind Quick Cartridge (Novagen) affinity column with binding 6His-Tag. SDS-PAGE showed a single band at 28 kDa (FIG. 2). The band was transferred onto the PVDF membrane and the N terminal amino acid was sequenced by Edams Hydrolysis, which shows that the first 15 amino acids on N-terminus were identical to those in SEQ ID NO: 2.

### EXAMPLE 6

### Preparation of Antibody Against N-acetylgalactosamine Transferase-28

The following specific N-acetylgalactosamine transferase-28 polypeptide was synthesized by a polypeptide synthesizer (PE-ABI): NH2-Met-Glu-Val-Tyr-Gly-Gly-Glu-Asn-Val-Glu-Leu-Gly-Ile-Arg-Val-COOH (SEQ ID NO:7). The polypeptide was conjugated with hemocyanin and bovine serum albumin (BSA) respectively to form two composites (See Avrameas et al., 1969, Immunochemistry, 6:43). 4 mg of hemocyanin-polypeptide composite was used to immunize rabbit together with Freund's complete adjuvant. The rabbit was re-immunized with the hemocyanin-polypeptide composite and Freund's incomplete adjuvent 15 days later. The titer of antibody in the rabbit sera was determined with a titration plate coated with 15 μg/ml BSA-polypeptide composite by ELISA. The total IgG was isolated from the sera of an antibody positive rabbit with Protein A-Sepharose. The polypeptide was bound to Sepharose 4B column activated by cyanogen bromide. The antibodies against the polypeptide were isolated from the total IgG by affinity chromatography. The immunoprecipitation approved that the purified antibodies could specifically bind to N-acetylgalactosamine transferase-28.

### EXAMPLE 7

### Application of the Polynucleotide Fragments of the Invention as Hybrid Probes

Selection of suitable oligonucleotides from the polynucleotide of the invention as hybrid probes can be versatilly applied. The probe could be used to determine the existence of polynucleotide of the invention or its homologous polynucleotide sequences by hybridization with genomic, or cDNA library of normal or clinical tissues from varied sources. The probes could be further used to determine whether polynucleotide of the invention or its homologous polynucleotide sequences are abnormally expressed in cells from normal or clinical tissues.

The aim of the following example is to select suitable oligonucletide fragments from SEQ ID NO:1 as hybird probes to apply in membrane hybridization to determine whether the polynucleotide of the invention or its homologous polynucleotide sequences exist in examined tissues. Membrane hybridization methods include dot hybridization, Southern blot, Northern blot, and replica hybridization. All these methods follow nearly the same steps after the polynucleotide samples are immobilized on membranes. These same steps are: membranes with samples immobilized there on are prehybridized in hybridization buffer not containing probes to block nonspecific binding sites of the samples on membranes. Then the prehybridization buffer is replaced by hybridization buffer containing labeled probes and incubation is continued at the appropriate temperature so probes hybridize with the target nucleotides. Free probes are washed off by a series of washing steps after the hybridization step. A high-stringency washing condition (relatively low salt concentration and high temperature) is applied in the example to reduce hybridization background and retain highly specific signal. Two types of probes are selected for the example: the first type are oligonucleotides identical or annealed to the SEQ ID NO:1; the second are oligonucleotides partially identical or partially annealed to SEQ ID NO:1. Dot blot method is applied in the example for immobilization of the samples on membrane. The strongest specific signal are produced by hybridization between the first type probes and samples after relatively strict membrane washing steps.

Selection of Probes

The general principles below should be followed for the selection of oligonucleotide fragments from the SEQ ID NO: 1 as hybrid probes:

1. The optimal length of probes should be between eighteen and fifty nucleotides.

2. GC amount should be between 30% and 70%, since nonspecific hybridization increases when GC amount is more than 70%.

3. There should be no complementary regions within the probes themselves.

4. Probes meeting the requirements above could be initially selected for further computer-aided sequence analysis, which includes homology comparison between the initially selected probes and its source sequence region (SEQ ID NO: 1), other known genomic sequences and their complements. Generally, the initial selected probes should not be used when they share fifteen identical continuous base pairs, or 85% homology with non-target region.

5. Whether the initially selected probes should be chosen for final application depend on further experimental confirmation.

The following two probes were selected and synthesized after the analysis above:

Probe one belongs to the first type probes, which is completely identical or annealed to the gene fragments of SEQ ID NO: 1(41 Nt);

5′-TGGAAGTCTACGGGGGCGAGAATGTGGAGCTTGGGATCAGG-3′ (SEQ ID NO: 8)

Probe two belongs to the second type probes which is a replaced or mutant sequence of the gene fragments of SEQ ID NO: 1, or of its complementary fragments (41 Nt):

5′-TGGAAGTCTACGGGGGCGAGCATGTGGAGCTTGGGATCAGG-3′ (SEQ ID NO: 9)

Any other frequently used reagents unlisted but involved in the following experimental steps and their preparation methods can be found in the reference: DNA PROBES, Keller and Manak, Stockton Press, 1989 (USA) or a more commonly used molecular cloning experimental handbook Molecular cloning (Sambrook et al., Acadimic Press, 1998, 2^{nd} edition).

Sample Preparation:

1, DNA Extraction from Fresh or Frozen Tissues

Steps: 1) move the fresh or newly thawy tissue (source tissue) onto an ice-incubated dish containing phosphate-buffered saline (PBS). Cut the tissue into small pieces with a scissor or an operating knife. Tissue should be kept damp throughout the operation. 2) mince the tissue by centrifugation at 2,000 g for 10 minutes. 3) resuspend the pellet (about 10ml/g) with cold homogenating buffer (0.25mol/l saccharose; 25 mmol/l Tris-HCl, pH7.5; 25 mmol/L NaCl; 25 mmol/L MgCl₂) at 4° C., and homogenate tissue suspension at full speed with an electronic homogenizer until completely smashed. 5) centrifuge at 1,000 g for 10 minutes. 6) resuspend the cell pellet (1-5 ml per 0.1 g initial tissue sample), and centrifuge at 1,000 g for 10 minutes. 7) resuspend the pellet with lysis buffer(1-5 ml per 0.1 g initial tissue sample), and continue to use the phenol extraction method.

2, Phenol Extraction of DNA

Steps: 1) wash cells with 1-10 ml cold PBS buffer and centrifuge at 1000 g for 10 minutes. 2) resuspend the precipitated cells with at least 100 μl cold cell lysis buffer (1×10⁸ cells/ml). 3) add SDS to a final concentration of 1%. Addition of SDS into the cell precipitation before cell resuspension will cause the formation of large cell aggreates which are difficult to disperse and homogenize, yield will be reduced. This phenomenon is especially severe when extracting more than 10⁷ cells. 5) incubate at 50° C. for an hour or shake gently overnight at 37° C. 6) add an equal volume of phenol: chloroform: isoamyl alcohol (25:24:1) to the DNA solution to be purified in a microcentrifuge tube, and centrifuge for 10 minutes. If the two phases are not clearly separated, the solution should be recentrifuged. 7) remove the aqueous phase to a new tube. 8) add an equal volume of chloroform: isoamyl alcohol (24:1) and centrifuge for 10 minutes. 9) remove the aqueous phase containing DNA to a new tube and then purify DNA by ethanol precipitation.

3, DNA Purification by Ethanol Precipitation

Steps: 1) add 1/10 vol of 2mol/L sodium acetate and 2 vol of cold 100% ethanol into the DNA solution, mix and place at −20° C. for an hour or overnight. 2) centrifuge for 10 minutes. 3) carefully spill the ethanol. 4) add 500 μl of cold 70% ethanol to wash the pellet and centrifuge for 5 minutes. 5) carefully spill the ethanol, add 500 μl cool ethanol to wash the pellets and centrifuge for 5 minutes. 6) carefully spill the ethanol and invert the tube on bibulous paper to remove remnant ethanol. Air dry for 10-15 minutes to evaporate ethanol on pellet surface. But notice not to dry the pellet completely since completely dry pellet is difficult to be dissolved again. 7) resuspend the DNA pellet with a small volume of TE or water. Spin at low speed or blow with a drip tube, and add TE gradually and mix until DNA is completely dissolved. Add 1 μl TE every 1-5×10⁶ cells.

The following 8-13 steps are applied only when contamination must be removed, otherwise go to step 14 directly. 8) add RNase A into the DNA solution to a final concentration of 100 μg/ml and incubate at 37° C. for 30 minutes. 9) add SDS and protease K to the final concentration of 0.5% and 100 μg/ml individually, and incubate at 37° C. for 30 minutes. 10) add an equal volume of phenol:chloroform:isoamyl alcohol (25:24:1), and centrifuge for 10 minutes. 11) carefully remove the aqueous phase and extract it with an equal volume of chloroform:isoamyl alcohol (24:1) and centrifuge for 10 minutes. 12) carefully remove out the aqueous phase, and add 1/10 vol of 2 mol/L sodium acetate and 2.5 vol of cold 100% ethanol, then mix and place at −20° C. for an hour. 13) wash the pellet with 70% ethanol and 100% ethanol, air dry and resuspend DNA as same as the steps 3-6. 14) determine the purity and production of DNA by A₂₆₀ and A₂₈₀ assay. 15) separate DNA sample into several portions and store at −20° C.

Preparation of Sample Membrane:

1) Take 4×2 pieces of nitrocellulose membrane (NC membrane) of desired size, and lightly mark out the sample dot sites and sample number with a pencil. Every probe needs two pieces of NC membrane, so that membranes could be washed under high stringency condition and stringency condition individually in the following experimental steps.

2) Pipette 15 μl of samples and control individually, dot them on the membrane, and dry at room tempreture.

3) Place the membranes on filter paper soaked in 0.1 mol/L NaOH, 1.5 mol/L NaCl, leave for 5 minutes (twice), and allow to dry. Transfer the membranes on filter paper soaked in 0.5 mol/L Tris-HCl (pH 7.0), 3 mol/L NaCl, leave for 5 minutes (twice), and allow to dry.

4) Place the membranes between clean filter paper, packet with aluminum foil, and vacuum dry at 60-80° C. for 2 hours.

Labeling of Probes

1) Add 3 μl probe (0.10D/10 μl), 2 μl kinase buffer, 8-10 μCi γ-³²P-dATP+2U Kinase, and add water to the final volume of 20 μl.

2) Incubate at 37° C. for 2 hours.

3) Add ⅕ vol bromophenol blue indicator (BPB).

4) Load that sample on Sephadex G-50 column.

5) Collect the first peak before the elution of ³²P-Probe ( monitor the eluting process by Monitor).

6) Five drops each tube and collect for 10-15 tubes.

7) Measure the isotope amount with liquid scintillator

8) The combined collection of the first peak is the prepared ³²P-Probe (the second peak is free γ-³²P-dATP)

Prehybridization

Place the sample membranes in a plastic bag, add 3-10 mg prehybrid buffer (10×Denhardt's; 6× SSC, 0.1 mg/ml CT DNA (calf thymus gland DNA), seal the bag, and shake on a 68° C. water bath for two hours (hybridization).

Cut off a corner of the plastic bag, add in prepared probes, seal the bag, and shake on a 42° C. water bath overnight.

Membrane Washing Under a High-Stringency Condition:

1) Take out the hybridized sample membranes

2) Wash the membranes with 2× SSC, 0.1% SDS at 40° C. for 15 minutes (twice).

3) Wash the membranes with 0.1× SSC, 0.1% SDS at 40° C. for 15 minutes (twice).

4) Wash the membranes with 0.1× SSC, 0.1% SDS at 55° C. for 30 minutes (twice), and dry at room temperature.

Membrane Washing Under A Low-Stringency Condition:

1) Take out the hybridized sample membranes.

2) Wash the membranes with 2× SSC, 0.1% SDS at 37° C. for 15 minutes (twice).

3) Wash the membranes with 0.1× SSC, 0.1% SDS at 37° C. for 15 minutes (twice).

4) Wash the membranes with 0.1× SSC, 0.1% SDS at 40° C. for 15 minutes (twice), and dry at room temperature.

X Ray Autoradiography

X Ray Autoradiograph at −70° C. (autoradiograph time varies according to radioactivity of the hybrid spot)

Experimental results:

In hybridization experiments carried out under low-stringency membrane washing condition, the radioactivity of all the above four probes hybrid spots shows no obvious difference; while in hybridization experiments carried out under high-stringency membrane washing condition, radioactivity of the hybrid spot by probe one is obviously stronger than the other three's. Thus probe one could be applied in qualitative and quantitive analysis of the existence and differential expression of said invented polynucleotide in different tissues.

We claim:

1. An isolated polypeptide -N-acetylgalactosamine transferase-28 comprising a polypeptide having the amino acid sequence of SEQ ID NO: 2, its active fragments, analogues and derivatives.

2. The polypeptide of claim 1 wherein amino acid sequences of said polypeptide, its analogues or derivatives have at least 95% identity with the amino acid sequence of SEQ ID NO: 2.

3. The polypeptide of claim 2 wherein said polypeptide is a polypeptide comprising the amino acid sequence of SEQ ID NO: 2.

4. An isolated polynucleotide selected from the group consisting of:
(a) the polynucleotide encoding a polypeptide having an amino acid sequence of SEQ ID NO: 2 or its fragment, analogue, derivative;
(b) the polynucleotide complementary to polynucleotide (a); and
(c) the polynucleotide sharing at least 70% identity to polynucleotide (a) or (b).

5. The polynucleotide of claim 4 comprising a polynucleotide encoding an amino acid sequence of SEQ ID NO:2.

6. The polynucleotide of claim 4 wherein the sequence of said polynucleotide comprises position 193-966 of SEQ ID NO:1 or position 1-1271 of SEQ ID NO:1.

7. A recombinant vector containing an exogenous polynucleotide which is constructed with the polynucleotide of any of claims 4-6 and plasmid, virus, or expression vector.

8. A genetically engineered host cell containing an exogenous polynucleotide which is selected form the group consisting of:
a) the host cell transformed or transfected by the recombinant vector of claim 7; and
(b) the host cell transformed or transfected by the polynucleotide of any of claims 4-6.

9. A method for producing a polypeptide having the activity of N-acetylgalactosamine transferase-28, which comprises the steps of:
(a) culturing the engineered host cell of claim 8 under the conditions suitable for expression of N-acetylgalactosamine transferase-28;
(b) isolating the polypeptides having the activity of N-acetylgalactosamine transferase-28 protein from the culture.

10. An antibody specifically which binds bound specifically with N-acetylgalactosamine transferase-28.

11. A compound simulating or regulating the activity or expression of the polypeptide which is the compound simulating, improving, antagonizing, or inhibiting the activity of N-acetylgalactosamine transferase-28.

12. The compound of claim 11 which is an antisense sequence of the polynucleotide sequence of SEQ ID NO: 1 or its fragment.

13. The use of the compound of claim 11 for regulating the activity of N-acetylgalactosamine transferase-28 in vivo or in vitro.

14. A method for detecting a disease related to the polypeptide of any of claims 1-3 or susceptibility thereof which comprises detecting the amount of expression of said polypeptide, or detecting the activity of said polypeptide, or detecting the nucleotide variant of the polynucleotide causing said abnormal expression or activity.

15. The use of the polypeptide of any of claims 1-3 for screening the mimetics, agonists, antagonists or inhibitors of N-acetylgalactosamine transferase-28; or for the identification of peptide spectrum.

16. The use of the nucleic acid molecule of any of claims 4-6 wherein it is used as primer in the nucleic acid amplification, or as probe in the hybridization reaction, or is used for manufacture of gene chip or microarray.

17. The use of the polypeptide, polynucleotide or compound of any of claims 1-6 and 11 wherein a safe and effective amount of said polypeptide, polynucleotide or its mimetics, agonists, antagonists or inhibitors are mixed with the pharmaceutically acceptable carrier to form the pharmaceutical composition for the diagnosis or treatment of diseases associated with the abnormality of N-acetylgalactosamine transferase-28.

18. The use of the polypeptide, polynucleotide or compound of any of claims 1-6 and 11 wherein said polypeptide, polynucleotide or compound are used for the manufacture of medicine for the treatment of developmental disorders, diseases caused by abnormal metabolism of immune system, and cancers.
